(19) United States
(12) Patent Application Publication
(10) Pub. No.: US 2007/0021765 A1
(43) Pub. Date: Jan. 25, 2007

(54) Ultrasonic treatment apparatus, probe for the same, and method of manufacturing the apparatus and the probe

(75) Inventor: Ryoji Sakai, Hachioji-shi (JP)

Correspondence Address: OSTROLENK FABER GERB & SOFFEN, 1180 AVENUE OF THE AMERICAS, NEW YORK, NY, 100368403, US

(73) Assignee: Olympus Medical Systems Corporation

(21) Appl. No.: 11/453,503

(22) Filed: Jun. 15, 2006

(30) Foreign Application Priority Data
Jun. 16, 2005 (JP) 2005-176554

(51) Int. Cl. A61B 17/32

(52) U.S. Cl. 606/169; 606/40; 606/207; 601/2

(57) ABSTRACT

There is provided an ultrasonic treatment apparatus including an ultrasonic oscillator which generates ultrasonic waves, a probe which is connected to the ultrasonic oscillator and transmits ultrasonic oscillations generated by the ultrasonic oscillator, and a treatment portion which is formed on the probe and treats a living tissue by the transmitted ultrasonic oscillations. The treatment portion has a cavitation suppressing portion formed to have such a shape that a pressure of a liquid in a vicinity of an external surface of the cavitation suppressing portion is greater than a saturation vapor pressure of the liquid in fluid analysis concerning ultrasonic oscillations in the liquid.

### CROSS-REFERENCE TO RELATED APPLICATIONS

This application is based upon and claims the benefit of priority from prior Japanese Patent Application No. 2005-176554, filed Jun. 16, 2005, the entire contents of which are incorporated herein by reference.

### BACKGROUND OF THE INVENTION

1. Field of the Invention

This invention relates to an ultrasonic treatment apparatus that performs treatment on a living tissue by using ultrasonic waves, such as an ultrasonic coagulation and incision apparatus and an ultrasonic aspiration apparatus.

2. Description of the Related Art

In prior art, ultrasonic treatment apparatuses that perform treatment on a living tissue by using ultrasonic waves have been used. For example, Jpn. Pat. Appln. KOKAI Pub. No. 2004-321606 discloses an ultrasonic coagulation and incision apparatus that coagulates and incises a living tissue. The ultrasonic coagulation and incision apparatus of Jpn. Pat. Appln. KOKAI Pub. No. 2004-321606 has an ultrasonic oscillator that generates ultrasonic oscillations. The ultrasonic oscillator is connected with a proximal end portion of an elongated probe that transmits ultrasonic oscillations, and a distal end portion of the probe is provided with a treatment portion that performs coagulation and incision of a living tissue by the transmitted ultrasonic oscillations. The treatment portion projects from a tip opening of a sheath covering the probe, and a distal end of the sheath is provided with a jaw that is opened and closed with respect to the treatment portion and holds a tissue in cooperation with the treatment portion. When a living tissue is treated by the ultrasonic coagulation and incision apparatus, the treatment portion and the jaw hold the tissue therebetween, ultrasonic oscillations generated by the ultrasonic oscillator are transmitted to the treatment portion through the probe, and the treatment portion coagulates and incises the held tissue.

If a living tissue is treated in the state where the treatment portion is immersed in liquid such as humor and blood, the tissue may be damaged due to cavitation occurring on the treatment portion. U.S. Pat. No. 6,790,216 discloses an ultrasonic coagulation and incision apparatus that suppresses occurrence of cavitation on a treatment portion. The ultrasonic coagulation and incision apparatus of U.S. Pat. No. 6,790,216 has almost the same structure as that of the ultrasonic coagulation and incision apparatus of Jpn. Pat. Appln. KOKAI Pub. No. 2004-321606, and also has a structure wherein an inclined portion that is inclined toward the tip is provided with the treatment portion on the opposite side of its holding surface facing the jaw. U.S. Pat. No. 6,790,216 also discloses that reducing the inclination angle of the inclined portion suppresses cavitation occurring in the treatment portion.

On the other hand, Jpn. Pat. Appln. KOKAI Pub. No. 2002-233533 discloses an ultrasonic aspiration apparatus that crushes and aspirates a living tissue. The ultrasonic aspiration apparatus of Jpn. Pat. Appln. KOKAI Pub. No. 2002-233533 has an ultrasonic oscillator, a probe, and a sheath, which are similar to those of the ultrasonic coagulation and incision apparatuses disclosed in Jpn. Pat. Appln. KOKAI Pub. No. 2004-321606 and U.S. Pat. No. 6,790,216. Further, a treatment portion that emulsifies and crushes a living tissue is formed on a tip portion of the probe of the ultrasonic aspiration apparatus disclosed in Jpn. Pat. Appln. KOKAI Pub. No. 2002-233533. Further, an aspiration channel that has an opening in the treatment portion and aspirates the crushed tissue is formed between the probe and the sheath. When a living tissue is treated by the ultrasonic aspiration apparatus, ultrasonic oscillations generated by the ultrasonic oscillator are transmitted to the treatment portion through the probe, the treatment portion emulsifies and crushes a living tissue, and the crushed tissue is aspirated through the aspiration channel.

### BRIEF SUMMARY OF THE INVENTION

An ultrasonic treatment apparatus according to an aspect of the present invention includes an ultrasonic oscillator which generates ultrasonic waves; a probe which is connected to the ultrasonic oscillator, and transmits ultrasonic oscillations generated by the ultrasonic oscillator; a treatment portion which is formed on the probe, and treats a living tissue by the transmitted ultrasonic oscillations, wherein the treatment portion has a cavitation suppressing portion formed to have a such shape that a pressure of a liquid in a vicinity of an external surface of the cavitation suppressing portion is greater than a saturation vapor pressure of the liquid in fluid analysis concerning ultrasonic oscillations in the liquid.

An ultrasonic treatment apparatus according to another aspect of the present invention includes an ultrasonic oscillator which generates ultrasonic waves; a probe which is connected to the ultrasonic oscillator, and transmits ultrasonic oscillations generated by the ultrasonic oscillator; and a treatment portion which is formed on the probe, and treats a living tissue by the transmitted ultrasonic oscillations, wherein the treatment portion has a cavitation promoting portion formed to have such a shape that a pressure of a liquid in a vicinity of an external surface of the cavitation promoting portion is equal to or less than a saturation vapor pressure of the liquid in fluid analysis concerning ultrasonic oscillations in the liquid.

A probe for an ultrasonic treatment apparatus according to another aspect of the present invention is connected to a ultrasonic oscillator which generates ultrasonic waves, transmits ultrasonic oscillations generated by the ultrasonic oscillator, and includes a treatment portion which is formed on the probe, treats a living tissue by the transmitted ultrasonic oscillations, and has a cavitation suppressing portion formed to have a such shape that a pressure of a liquid in a vicinity of an external surface of the cavitation suppressing portion is greater than a saturation vapor pressure of the liquid in fluid analysis concerning ultrasonic oscillations in the liquid.

A probe for an ultrasonic treatment apparatus according to another aspect of the present invention is connected to a ultrasonic oscillator which generates ultrasonic waves, transmits ultrasonic oscillations generated by the ultrasonic oscillator, and includes a treatment portion which is formed on the probe, treats a living tissue by the transmitted ultrasonic oscillations, and has a cavitation promoting portion formed to have such a shape that a pressure of a liquid in a vicinity of an external surface of the cavitation promoting portion is equal to or less than a saturation vapor pressure of the liquid in fluid analysis concerning ultrasonic oscillations in the liquid.

A method of manufacturing a probe for ultrasonic treatment apparatus according to another aspect of the present invention includes preparing a predetermined shape model for at least part of a treatment portion which treats a living tissue by ultrasonic oscillations; obtaining, by fluid analysis concerning ultrasonic oscillations in a liquid, a pressure distribution of the liquid with respect to the shape model; changing a shape of the shape model such that a pressure of at least part of portions where the pressure is equal to or less than a saturation vapor pressure of the liquid in the pressure distribution becomes greater than the saturation vapor pressure of the liquid; alternately repeating the obtaining the pressure distribution of the liquid and the changing the shape of the shape model; and forming the treatment portion to have a shape of the shape model.

A method of manufacturing a probe for ultrasonic treatment apparatus according to another aspect of the present invention includes preparing a predetermined shape model for at least part of a treatment portion which treats a living tissue by ultrasonic oscillations; obtaining, by fluid analysis concerning ultrasonic oscillations in a liquid, a pressure distribution of the liquid with respect to the shape model; changing a shape of the shape model such that a pressure of at least part of portions where the pressure is greater than a saturation vapor pressure of the liquid in the pressure distribution becomes less than the saturation vapor pressure of the liquid; alternately repeating the obtaining the pressure distribution of the liquid and the changing the shape of the shape model; and forming the treatment portion to have a shape of the shape model.

A method of manufacturing an ultrasonic treatment apparatus according to another aspect of the present invention includes a method of manufacturing a probe, including preparing a predetermined shape model for at least part of a treatment portion which treats a living tissue by ultrasonic oscillations; obtaining, by fluid analysis concerning ultrasonic oscillations in a liquid, a pressure distribution of the liquid with respect to the shape model; changing a shape of the shape model such that a pressure of at least part of portions where the pressure is equal to or less than a saturation vapor pressure of the liquid in the pressure distribution becomes greater than the saturation vapor pressure of the liquid; alternately repeating the obtaining the pressure distribution of the liquid and the changing the shape of the shape model; and forming the treatment portion to have a shape of the shape model.

A method of manufacturing an ultrasonic treatment apparatus according to another aspect of the present invention includes a method of manufacturing a probe, including preparing a predetermined shape model for at least part of a treatment portion which treats a living tissue by ultrasonic oscillations; obtaining, by fluid analysis concerning ultrasonic oscillations in a liquid, a pressure distribution of the liquid with respect to the shape model; changing a shape of the shape model such that a pressure of at least part of portions where the pressure is greater than a saturation vapor pressure of the liquid in the pressure distribution becomes less than the saturation vapor pressure of the liquid; alternately repeating the obtaining the pressure distribution of the liquid and the changing the shape of the shape model; and forming the treatment portion to have a shape of the shape model.

### BRIEF DESCRIPTION OF THE SEVERAL VIEWS OF THE DRAWING

The accompanying drawings, which are incorporated in and constitutes a part of the specification, illustrate embodiments of the invention, and together with the general description given above and the detailed description of the embodiments given below, serve to explain the principles of the invention.

FIG. 1 is a side view of an ultrasonic coagulation and incision apparatus according to a first embodiment of the present invention.

FIG. 2 is a perspective view of a tip portion of the ultrasonic coagulation and incision apparatus according to the first embodiment of the present invention.

FIG. 3A is a perspective view of a probe of the ultrasonic coagulation and incision apparatus according to the first embodiment of the present invention, in an oscillation state toward a tip side.

FIG. 3B is a perspective view of the probe of the ultrasonic coagulation and incision apparatus according to the first embodiment of the present invention, in an oscillation state toward a rear end side.

FIG. 4 is a perspective view of an initial three-dimensional model of a treatment portion, in a method of designing the treatment portion of the ultrasonic coagulation and incision apparatus according to the first embodiment of the present invention.

FIG. 5 is a pressure distribution diagram for the initial three-dimensional model prepared as a result of fluid analysis, in the method of designing the treatment portion of the ultrasonic coagulation and incision apparatus according to the first embodiment of the present invention.

FIG. 6 is a velocity distribution diagram for the initial three-dimensional model prepared as a result of fluid analysis, in the method of designing the treatment portion of the ultrasonic coagulation and incision apparatus according to the first embodiment of the present invention.

FIG. 7 is a perspective view of a final three-dimensional model of the treatment portion, in the method of designing the treatment portion of the ultrasonic coagulation and incision apparatus according to the first embodiment of the present invention.

FIG. 8 is a pressure distribution diagram of the final three-dimensional model prepared as a result of fluid analysis, in the method of designing the treatment portion of the ultrasonic coagulation and incision apparatus according to the first embodiment of the present invention.

FIG. 9 is a perspective view of a probe of an ultrasonic coagulation and incision apparatus according to a modification of the first embodiment of the present invention, in an oscillation state.

FIG. 10 is a side view of an ultrasonic aspiration apparatus according to a second embodiment of the present invention.

FIG. 11 is a perspective view of an initial three-dimensional model of a treatment portion, in a method of designing the treatment portion of the ultrasonic coagulation and incision apparatus according to the second embodiment of the present invention.

FIG. 12 is a pressure distribution diagram for the initial three-dimensional model prepared as a result of fluid analysis, in the method of designing the treatment portion of the ultrasonic coagulation and incision apparatus according to the second embodiment of the present invention.

FIG. 13 is a perspective view of a final three-dimensional model of the treatment portion, in the method of designing the treatment portion of the ultrasonic coagulation and incision apparatus according to the second embodiment of the present invention.

FIG. 14 is a pressure distribution diagram for the initial three-dimensional model prepared as a result of fluid analysis, in the method of designing the treatment portion of the ultrasonic coagulation and incision apparatus according to the second embodiment of the present invention.

FIG. 15 is a diagram illustrating values of a drag coefficient C_{D} with respect to Reynolds numbers Re for various forms.

### DETAILED DESCRIPTION OF THE INVENTION

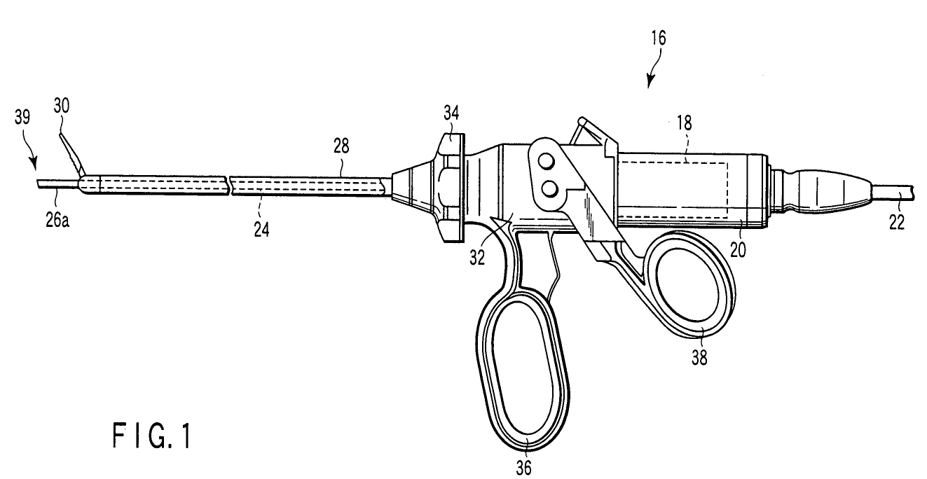

A first embodiment of the present invention is described with reference to FIGS. 1 to 8. The ultrasonic treatment apparatus according to the first embodiment is an ultrasonic coagulation and incision apparatus 16 that suppresses occurrence of cavitation. As shown in FIG. 1, the ultrasonic coagulation and incision apparatus 16 has an ultrasonic oscillator 18 that generates ultrasonic oscillations. The ultrasonic oscillator 18 is accommodated in a cylindrical cover 20. A code 22 to supply electric power to the ultrasonic oscillator 18 is extended out of a proximal end portion of the cylindrical cover 20. Further, an output end on a tip portion of the ultrasonic oscillator 18 is connected with a proximal end portion of a probe 24 transmitting the ultrasonic oscillations and having an elongated straight shape. A treatment portion 26a that coagulates and incises a living tissue by the transmitted ultrasonic oscillations is formed on a tip portion of the probe 24.

Further, the probe 24 is covered with a sheath 28. A jaw 30 that is opened and closed with respect to the treatment portion 26a and holds a living tissue in cooperation with the treatment portion 26a is provided on a tip portion of the sheath 28. On the other hand, a proximal end portion of the sheath 28 is connected with an operation main body 32 such that the sheath 28 is rotatable around its central axis. A rotary knob 34 to rotate the sheath 28 is provided on the proximal end portion of the sheath 28. Further, a fixed handle 36 and a movable handle 38 to open and close the jaw 30 are provided on the operation main body 32. Specifically, the movable handle 38 is pivotally supported by the operation main body 32 such that the movable handle 38 is openable and closable with respect to the fixed handle 36, and is pivotally supported by a proximal end portion of an operation rod located in the operation main body 32. The operation rod is inserted through the operation main body 32 and the sheath 28 such that the operation rod is movable back and forth, and the tip portion of the operation rod is connected to a proximal end of the jaw 30. Further, the operation rod is moved back and forth by opening and closing the movable handle 38 with respect to the fixed handle 36, and thereby the jaw 30 is opened and closed with respect to the treatment portion 26a.

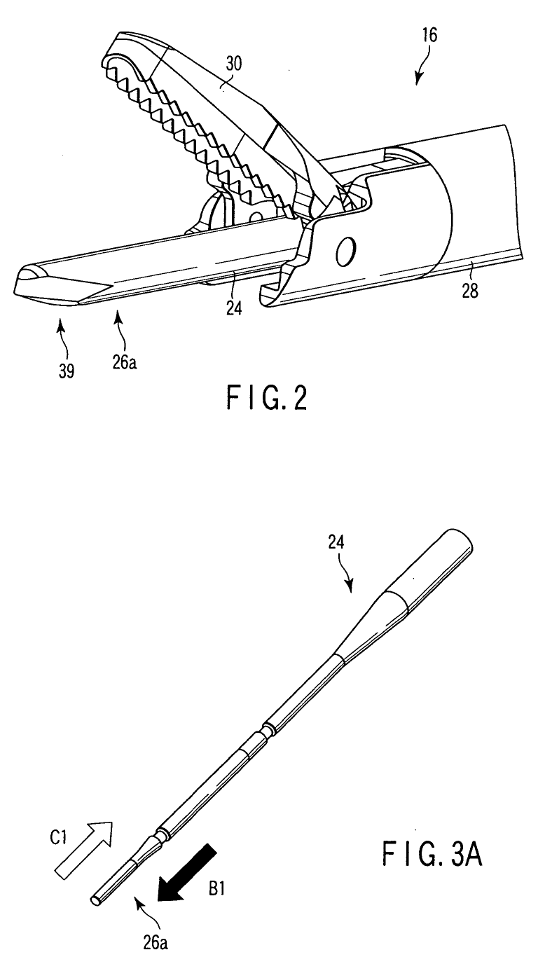

The treatment portion 26a illustrated in FIG. 2 according to the first embodiment has a form that suppresses generation of cavitation in the case where it is oscillated by ultrasonic waves in liquid such as humor and blood. The following is explanation of a method of designing the treatment portion 26a.

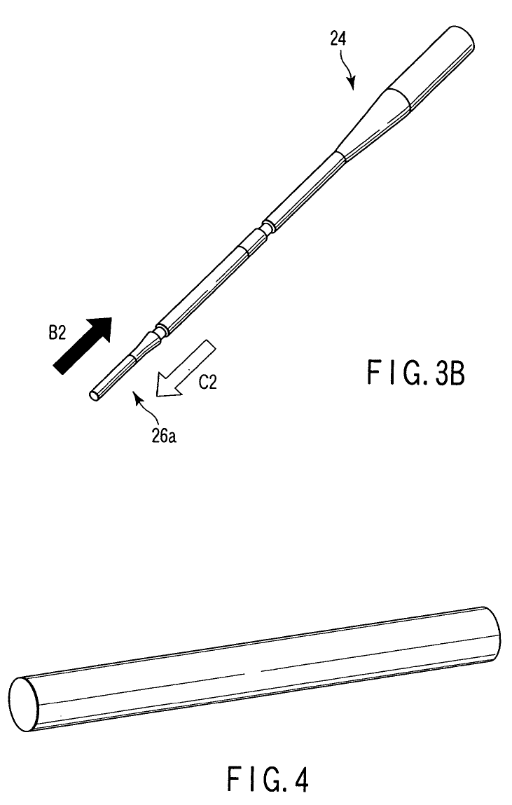

Step 1: Preparation of initial three-dimensional model An initial three-dimensional model is prepared for the probe 24 as shown in FIGS. 3A and 3B. In the first embodiment, a conventional ultrasonic coagulation and incision probe is adopted as the initial three-dimensional model.

Step 2: Fluid analysis based on the three-dimensional model

Fluid analysis is performed for the case where the probe 24 is oscillated in liquid by ultrasonic waves.

The probe 24 is longitudinally oscillated in liquid in its longitudinal direction with a predetermined amplitude and period. Specifically, the probe 24 repeats oscillation toward the tip side illustrated by arrow B1 in FIG. 3A, and oscillation toward the proximal end side shown by arrow B2 in FIG. 3B. In the first embodiment, analysis is performed by using a coordinate system fixed on the probe 24. In the coordinate system, the probe 24 is located in a state of rest in a liquid field that oscillates with a predetermined amplitude and period, such that the longitudinal direction of the probe 24 coincides with the oscillation direction of the liquid. Specifically, the liquid repeats oscillation toward the proximal end side illustrated by arrow C1 in FIG. 3A, and oscillation toward the tip side illustrated by arrow C2 in FIG. 3B.

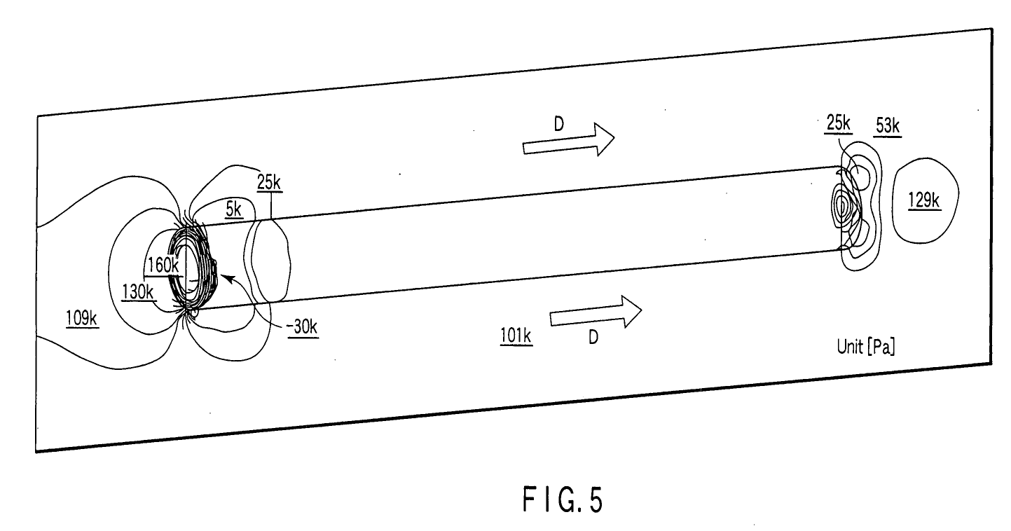

In the first embodiment, fluid analysis is performed only with respect to the treatment portion 26a in the tip portion of the probe 24, to reduce analysis time in the fluid analysis. Specifically, a three-dimensional model of the treatment portion 26a whose both end portions have the same shape as those of the treatment portion 26a is prepared on the basis of the three-dimensional model of the probe 24. FIG. 4 illustrates an example of the prepared three-dimensional model of the treatment portion 26a. The three-dimensional model of the treatment portion 26a corresponds to a cylindrical shape adopted in the treatment portion 26a of a conventional ultrasonic coagulation and incision probe 24.

Then, prepared is a liquid field model for a half period of the above liquid field that oscillates in the one direction with a predetermined amplitude and period, that is, a liquid field model whose amplitude increases from 0 to its maximum amplitude and decreases from the maximum amplitude to 0 with a predetermined period, and then amplitude increases again without decrease. The three-dimensional model of the treatment portion 26a is located in a state of rest in the liquid field model such that the longitudinal direction of the three-dimensional model coincides with the oscillation direction of the liquid, and then fluid analysis is performed. In the three-dimensional model of the treatment portion 26a, at an end portion on an upstream side of the oscillation direction of the liquid field model, behavior when the treatment portion 26a is oscillated toward the tip side is analyzed. At an end portion on a downstream side of the oscillation direction, behavior when the treatment portion 26a is oscillated toward the proximal end side is analyzed. In fluid analysis, a pressure distribution and a velocity distribution of the liquid field model are calculated.

Based on the pressure distribution of the liquid field model, occurrence of cavitation is analyzed. Generally, cavitation occurs if the liquid reaches its saturation vapor pressure. For example, water reaches its saturation vapor pressure and cavitation occurs, when its temperature is increased to 100° C. under atmospheric pressure (101.3 kPa), and when its pressure is reduced to 2 kPa under standard temperature (20° C.). If a living tissue is treated in liquid with the treatment portion 26a, it is expected that cavitation occurs in portions corresponding to portions of the liquid, whose pressures are reduced to its saturation vapor pressure in the liquid field model.

FIG. 5 illustrates an example of a pressure distribution diagram prepared as a result of the fluid analysis. In FIG. 5, the oscillation direction of the liquid is indicated by arrows D. Water at standard temperature (20° C.) is selected as the liquid of the liquid field model. It is expected that cavitation occurs in portions where the pressure is equal to or less than the saturation vapor pressure (2 kPa) in the liquid field model. As shown in FIG. 5, the pressure of the liquid field model is 2 kPa or less in the vicinity of an edge of the upstream end portion in the three-dimensional model of the treatment portion 26a. Thus, if a living tissue is treated in liquid with the treatment portion 26a, it is expected that cavitation occurs in the vicinity of an edge portion of the treatment portion 26a when the treatment portion 26a is oscillated toward the tip side. In a corresponding actual experiment, it has been verified that cavitation occurs in an edge portion of the treatment portion 26a when it is oscillated toward the tip side if a living tissue is treated in liquid with the treatment portion 26a.

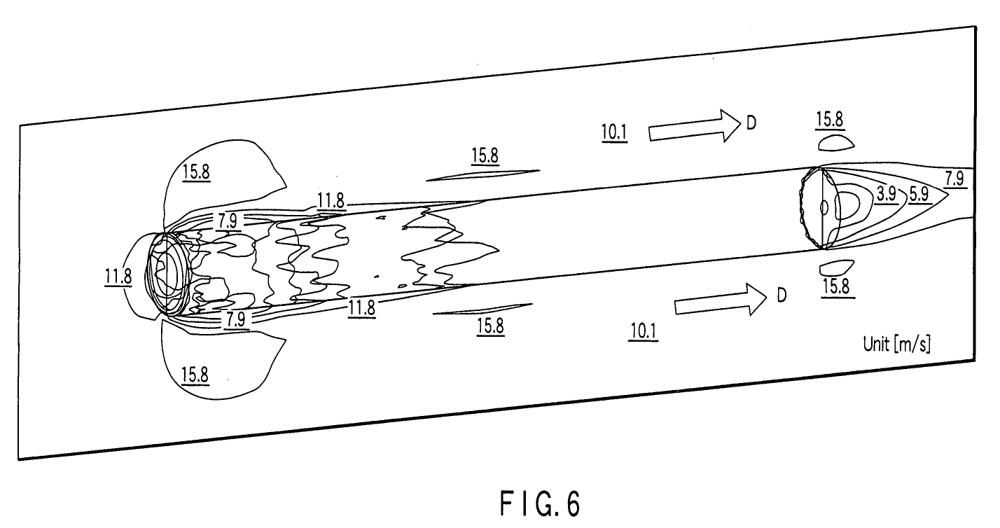

FIG. 6 illustrates an example of a velocity distribution diagram prepared as a result of the fluid analysis. In FIG. 6, the oscillation direction of the liquid is indicated by arrows D. As shown in FIG. 6, it is understood that the velocity of the liquid of the liquid field model converges on one point in a downstream end portion of the three-dimensional model of the treatment portion 26a. Specifically, cavitation generated in an edge portion of the treatment portion 26a when the treatment portion 26a is oscillated toward the tip side is assumed to move from the edge portion of the treatment portion 26a toward the tip side when the treatment portion 26a is oscillated toward the proximal end side. Also in a corresponding actual experiment, it has been verified that cavitation moves from the edge portion of the treatment portion 26a toward the tip side in oscillation of the treatment portion 26a toward the proximal end side.

Step 3: Change in shape of the three-dimensional model

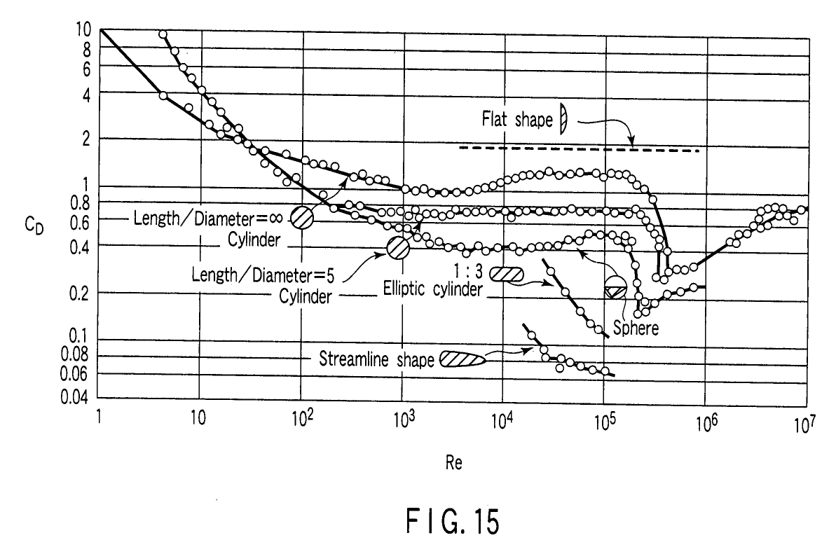

The shape of the three-dimensional model of the treatment portion 26a is changed such that the pressures of portions where the pressure is equal to or less than the saturation vapor pressure of the liquid become greater than the saturation vapor pressure. In the first embodiment, the shape of portions of the three-dimensional model in the vicinity of portions of the liquid field model, where the pressure is equal to or less than the saturation vapor pressure of the liquid, is changed to a shape having a smaller drag coefficient. If the drag coefficient is small, a pressure gradient becomes gentler, and decrease in the pressure of the liquid in the liquid field model is suppressed. Specifically, in FIG. 5, the pressure of the liquid field model in the vicinity of the edge portion of the upstream end portion in the three-dimensional model of the treatment portion 26a is equal to or less than the saturation vapor pressure (2 kPa). The shape of the edge portion is changed to a streamline shape having a small drag coefficient. As a matter of course, the shape of the edge portion of the downstream end is also changed in conformity with the modification of the edge portion of the upstream end. FIG. 15 illustrates values of drag coefficient C_{D} for Reynolds numbers Re for various shapes.

Step 4: Repetition of fluid analysis based on the three-dimensional model and change in shape of the three-dimensional model

The fluid analysis based on the three-dimensional model of Step 2 and the change in shape of the three-dimensional model of Step 3 are alternately repeated.

Step 5: Determination of final three-dimensional model

When the portions in the liquid field model where the pressure is reduced to the saturation vapor pressure of the liquid are almost disappeared, change in shape of the three-dimensional model is ended, and the final three-dimensional model of the treatment portion 26a is determined.

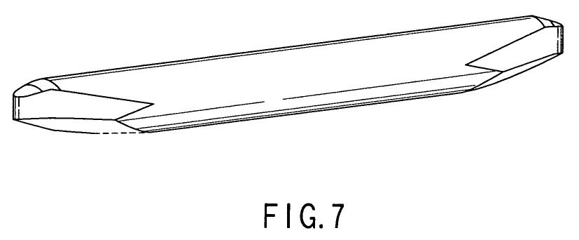

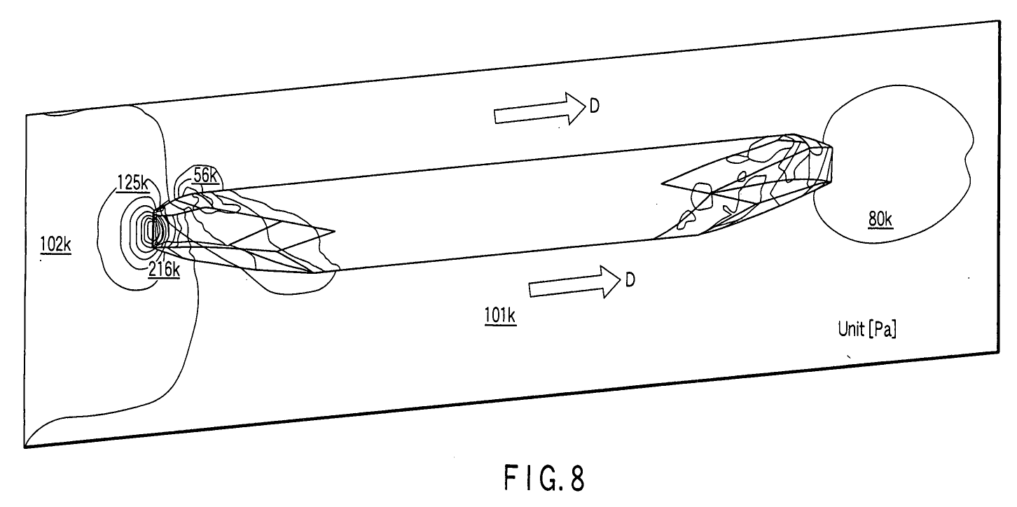

FIG. 7 illustrates an example of the final three-dimensional model of the treatment portion 26a. As shown in FIG. 7, the three-dimensional model of the treatment portion 26a has a shape close to a streamline shape. FIG. 8 is a pressure distribution diagram prepared as a result of the fluid analysis of the three-dimensional model of the treatment portion 26a. In FIG. 8, the oscillation direction of the liquid is indicated by arrows D. As shown in FIG. 8, the portions where the pressure is equal to or less than the saturation vapor pressure (2 kPa) have almost been disappeared. Therefore, it is expected that occurrence of cavitation is suppressed in the case where a living tissue is treated in liquid with the treatment portion 26a. Also in a corresponding actual experiment, it has been verified that occurrence of cavitation is suppressed if a living tissue is treated in liquid with the treatment portion 26a.

As described above, according to the first embodiment, the tip portion of the treatment portion 26a is a cavitation suppressing portion 39 that suppresses occurrence of cavitation.

Next, operation of the ultrasonic coagulation and incision apparatus 16 according to the first embodiment is explained. When a living tissue is treated with the ultrasonic coagulation and incision apparatus 16, the treatment portion 26a and the jaw 30 hold the tissue therebetween, ultrasonic oscillations generated by the ultrasonic oscillator 18 are transmitted to the treatment portion 26a through the probe 24, and then the treatment portion 26a coagulates and incises the held tissue. In this process, although the treatment portion 26a may be immersed in liquid such as humor and blood, the pressure gradient of the liquid is gentle in the vicinity of the external surface of the treatment portion 26a, and thus the pressure of the liquid rarely becomes equal to or less than the saturation vapor pressure of the liquid. Therefore, occurrence of cavitation in the treatment portion 26a is suppressed.

Therefore, the ultrasonic coagulation and incision apparatus 16 of the first embodiment has the following effects. The treatment portion 26a of the first embodiment is formed to have a shape such that the pressure in the vicinity of the external surface of the treatment portion 26a is greater than the saturation vapor pressure of the liquid, in the fluid analysis concerning the ultrasonic oscillation in the liquid. Further, occurrence of cavitation in the treatment portion 26a when the treatment portion 26a coagulates and incises a living tissue in liquid is actually suppressed, and an optimum cavitation state in coagulation and incision is realized.

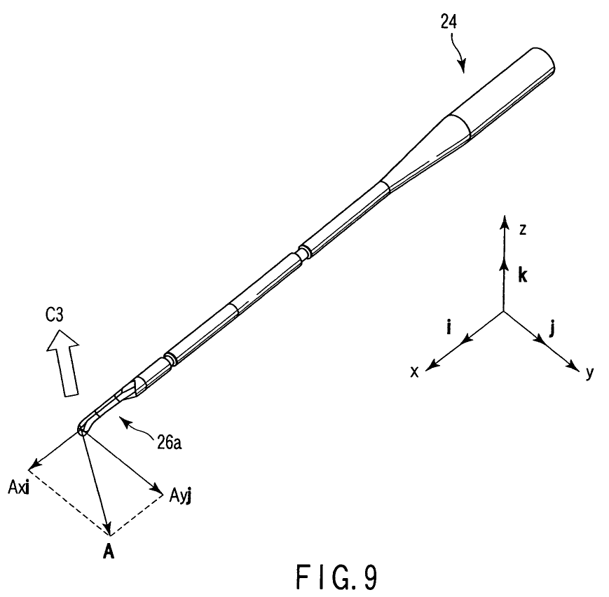

A modification of the first embodiment of the present invention is explained with reference to FIG. 9. In the modification, an optimum cavitation state is realized for the treatment portion 26a that is oscillated in a three-dimensional manner.

In the probe 24 having a straight shape as in the first embodiment, the treatment portion 26a is oscillated in a one-dimensional manner. On the other hand, in a common probe 24, the treatment portion 26a is oscillated in a three-dimensional manner. Specifically, an amplitude vector of the treatment portion 26a is represented as follows by using vector components in X, Y and Z axis directions.
A=Ax·i+Ay·j+Az·k

i, j, k: unit vectors of respective axis directions

Ax, Ay, Az: sizes of amplitudes of respective axis directions

The sizes of amplitudes of the respective axis directions can be calculated by numerical analysis. A liquid field model used for fluid analysis in design of the treatment portion 26a is prepared on the basis of the sizes of the amplitudes of the respective axis directions.

For example, in the probe 24 having a curved shape as shown in FIG. 9, the treatment portion 26a is oscillated in a two-dimensional manner. In this case, sizes of amplitudes of the X axis and the Y axis are calculated by numerical analysis, and the liquid field model used for the fluid analysis in the method of designing the treatment portion 26a is prepared as indicated by arrow C3.

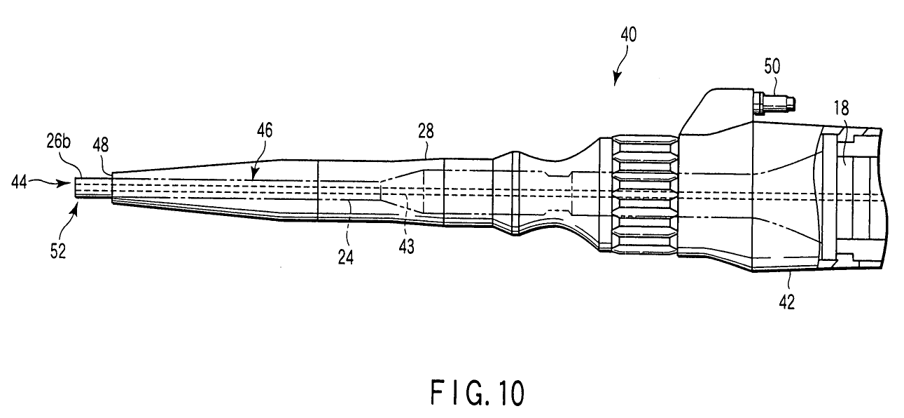

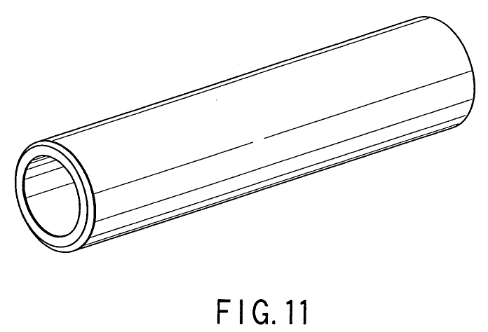

FIGS. 10 to 14 illustrate a second embodiment of the present invention. Structures having similar functions as those in the first embodiment are denoted by the same reference numerals as those in the first embodiment, and explanation thereof is omitted. The ultrasonic treatment apparatus of the second embodiment is an ultrasonic aspiration apparatus 40 that crushes and aspirates a living tissue. As shown in FIG. 10, an ultrasonic oscillator 18 of the ultrasonic aspiration apparatus 40 is accommodated in a hand piece 42. Further, an output end of the ultrasonic oscillator 18 is connected with a proximal end portion of a probe 24, and a treatment portion 26b that emulsifies and crushes a living tissue by transmitted ultrasonic oscillations is formed on a tip portion of the probe 24.

Further, an aspiration channel 43 to aspirate the crushed tissue is formed through the probe 24 and the ultrasonic oscillator 18 in the longitudinal direction of the probe 24 and the ultrasonic oscillator 18. A tip portion of the aspiration channel 43 is opened at the treatment portion 26b and forms an aspiration opening portion 44. A proximal end portion of the aspiration channel 43 communicates with an aspiration connecter formed in the hand piece 42, and the aspiration connecter is connected to an aspiration apparatus.

Further, the probe 24 is covered with a sheath 28, and a clearance between the probe 24 and the sheath 28 forms a liquid conveying channel 46 to convey liquid. A tip portion of the liquid conveying channel 46 is annularly opened between the tip portion of the sheath 28 and the probe 24 and forms a liquid conveying opening portion 48. A proximal end portion of the liquid conveying channel 46 communicates with a liquid conveying connecter 50 provided on the hand piece, and the liquid conveying connecter 50 is connected with a liquid conveying apparatus.

The treatment portion 26b of the embodiment has a shape that promotes occurrence of cavitation when the treatment portion 26b is oscillated by ultrasonic waves in liquid such as a physiological saline solution. A method of designing the treatment portion 26b is explained below. Explanation of steps similar to those in the designing method according to the first embodiment is omitted.

Step 1: Preparation of initial three-dimensional model

In the second embodiment, a conventional ultrasonic aspiration probe is adopted as an initial three-dimensional model.

Step 2: Fluid analysis based on the three-dimensional model

As shown in FIG. 11, an almost cylindrical three-dimensional model of the treatment portion 26b is prepared on the basis of a three-dimensional model of the probe 24. Both ends of the three-dimensional model of the treatment portion 26b have the same shape as those of the treatment portion 26b.

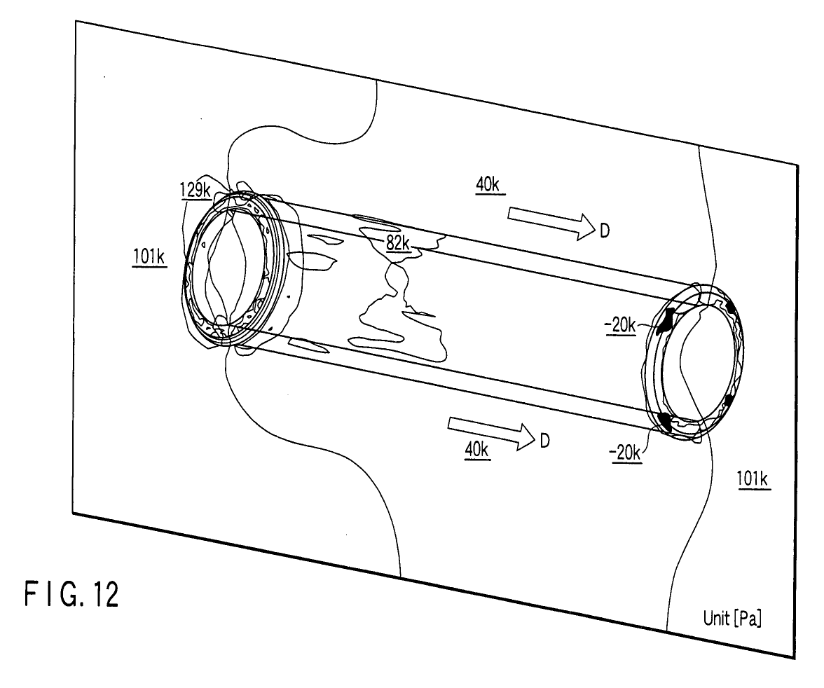

FIG. 12 illustrates an example of a pressure distribution diagram prepared as a result of fluid analysis. In FIG. 12, the oscillation direction of the liquid is indicated by arrows D. As shown in FIG. 12, the pressure of the liquid field model is 2 kPa or less in the vicinity of an annular end surface of a downstream end portion in the three-dimensional model of the treatment portion 26b. Therefore, if a living tissue is actually treated in liquid with the treatment portion 26b, it is expected that cavitation occurs in the vicinity of the annular end surface of the treatment portion 26b when the treatment portion 26b is oscillated toward the rear end side.

Step 3: Change in shape of the three-dimensional model

The shape of the three-dimensional model of the treatment portion 26b is changed such that, with respect to portions where cavitation is required to occur when a living tissue is treated in liquid with the treatment portion 26b, the pressure in corresponding portions in the liquid field model is equal to or less than the saturation vapor pressure. In the second embodiment, the shape of the three-dimensional model in the vicinity of portions of the liquid field model where the pressure is required to be equal to or lower than the saturation vapor pressure of the liquid is changed to a shape having a large drag coefficient. Increasing a drag coefficient makes a pressure gradient steep, and increases reduction in pressure of the liquid in the liquid field model. Specifically, with reference to FIG. 12, if the pressure of the portions of the liquid field model in the vicinity of the annular end surfaces in the both end portions of the three-dimensional model of the treatment portion 26b is required to be equal to or lower than the saturation vapor pressure (2 kPa), the shape of the three-dimensional model is changed such that the both end portions of the external peripheral portions in the three-dimensional model of the treatment portion 26b have the same flange shape to increase the drag coefficient with respect to the oscillation direction of the liquid field.

Step 4: Repetition of fluid analysis based on the three-dimensional model and the change in shape of the three-dimensional model Step 5: Determination of final three-dimensional model

When the pressure in the portions in the liquid field model, which is required to be equal to or less than the saturation vapor pressure, has become equal to or less than the saturation vapor pressure, change in shape of the three-dimensional model is ended, and a final three-dimensional model of the treatment portion 26b is determined.

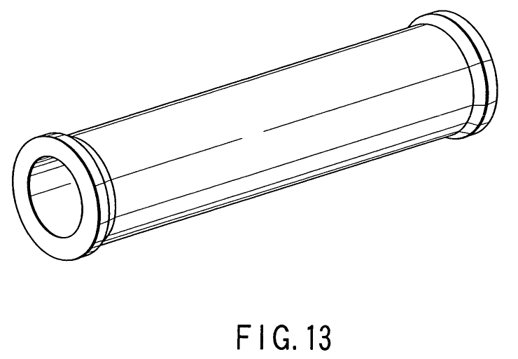

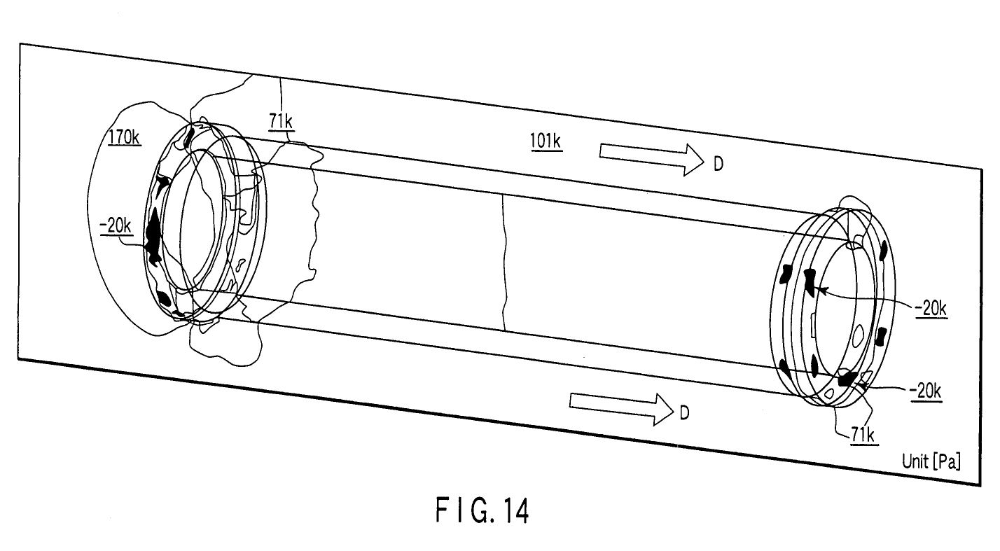

FIG. 13 illustrates an example of the final three-dimensional model of the treatment portion 26b. As shown in FIG. 13, the three-dimensional model of the treatment portion 26b has a shape in which the end portions have a flange shape. FIG. 14 is a pressure distribution diagram prepared as a result of the fluid analysis with respect to the three-dimensional model of the treatment portion 26b. In FIG. 14, the oscillation direction of the liquid is indicated by arrows D. As shown in FIG. 14, portions where the pressure is equal to or less than the saturation vapor pressure (2 kPa) are formed in the vicinity of the annular end surfaces in the both end portions of the three-dimensional model of the treatment portion 26b. Therefore, it is expected that occurrence of cavitation is promoted in the case where a living tissue is treated in liquid with the treatment portion 26b. Also in a corresponding actual experiment, it has been verified that occurrence of cavitation is promoted if a living tissue is treated in liquid with the treatment portion 26b.

As described above, according to the second embodiment, the tip portion of the treatment portion 26a is a cavitation promoting portion 52 that promotes occurrence of cavitation.

Next, operation of the ultrasonic aspiration apparatus 40 according to the second embodiment is explained. When a living tissue is treated with the ultrasonic aspiration apparatus 40, an aspiration apparatus and a liquid conveying apparatus are connected to the aspiration connecter and the liquid conveying connecter 50, respectively. Then, the treatment portion 26b and the tissue are immersed in liquid such as physiological saline solution by conveying the liquid through the liquid conveying opening portion 48. In this state, ultrasonic oscillations generated by the ultrasonic oscillator 18 are transmitted to the treatment portion 26b through the probe 24, and the treatment portion 26b is pressed onto the tissue to emulsify and crush the tissue. In this process, the treatment portion 26b is immersed in liquid such as physiological saline solution, the pressure gradient of the liquid is steep in the vicinity of the external surface of the treatment portion 26b, and thus the pressure of the liquid becomes equal to or less than the saturation vapor pressure of the liquid. Therefore, occurrence of cavitation in the treatment portion 26b is promoted. Thus, emulsification and crushing of the tissue are effectively performed. The emulsified and crushed tissue is aspirated through the aspiration channel 43 via the aspiration opening portion 44.

Therefore, the ultrasonic aspiration apparatus 40 of the second embodiment has the following effects. The treatment portion 26b of the second embodiment is formed to have a shape such that the pressure in the vicinity of the external surface of the treatment portion 26b is equal to or less than the saturation vapor pressure of the liquid, in the fluid analysis concerning the ultrasonic oscillation in the liquid. Further, occurrence of cavitation in the treatment portion 26b when the treatment portion 26b emulsifies and crushes a living tissue in liquid is actually promoted, and an optimum cavitation state in emulsification and crushing is realized.

The following is explanation of a modification of the second embodiment of the present invention. The treatment portion 26b in the modification has a shape such that occurred cavitation moves toward the tissue when the treatment portion 26b is oscillated by ultrasonic waves in liquid such as physiological saline solution.

In a method of designing the treatment portion 26b of the modification, in a step of changing the shape of the three-dimensional model, the shape of the three-dimensional model of the treatment portion 26b is changed such that the direction of velocity of the liquid in portions of the liquid field model where the pressure is equal to or less than the saturation vapor pressure of the liquid corresponds to the direction from the treatment portion 26b toward the tissue in treatment on the tissue. Specifically, with reference to FIG. 14, the shape of the three-dimensional model is changed such that the direction of velocity of the liquid in the portions of the liquid field model, where the pressure is equal to or less than the saturation vapor pressure (2 kPa) of the liquid, in the vicinity of the end portions in the three-dimensional model of the treatment portion 26b corresponds to the direction from the treatment portion 26b toward the tissue in treatment on the tissue, that is, an outward longitudinal direction of the treatment portion 26b.

When a living tissue is treated with the ultrasonic aspiration apparatus 40 according to the modification, cavitation generated by the treatment portion 26b moves toward the tissue, and reaches the tissue to promote emulsification and crushing of the tissue. Therefore, according to the treatment portion 26b of the modification, cavitation generated by the treatment portion 26b efficiently reaches the tissue, and promotes emulsification and crushing of the tissue.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic treatment apparatus comprising:
an ultrasonic oscillator which generates ultrasonic waves;
a probe which is connected to the ultrasonic oscillator, and transmits ultrasonic oscillations generated by the ultrasonic oscillator;
a treatment portion which is formed on the probe, and treats a living tissue by the transmitted ultrasonic oscillations,
wherein the treatment portion has a cavitation suppressing portion formed to have a such shape that a pressure of a liquid in a vicinity of an external surface of the cavitation suppressing portion is greater than a saturation vapor pressure of the liquid in fluid analysis concerning ultrasonic oscillations in the liquid.

2. An ultrasonic treatment apparatus according to claim 1, wherein
the cavitation suppressing portion is formed to have a shape having a small drag coefficient.

3. An ultrasonic treatment apparatus according to claim 1 used to coagulate and incise the living tissue.

4. An ultrasonic treatment apparatus according to claim 3, further comprising:
a jaw which is opened and closed with respect to the treatment portion, and holds the tissue in cooperation with the treatment portion.

5. An ultrasonic treatment apparatus comprising:
an ultrasonic oscillator which generates ultrasonic waves;
a probe which is connected to the ultrasonic oscillator, and transmits ultrasonic oscillations generated by the ultrasonic oscillator; and
a treatment portion which is formed on the probe, and treats a living tissue by the transmitted ultrasonic oscillations,
wherein the treatment portion has a cavitation promoting portion formed to have such a shape that a pressure of a liquid in a vicinity of an external surface of the cavitation promoting portion is equal to or less than a saturation vapor pressure of the liquid in fluid analysis concerning ultrasonic oscillations in the liquid.

6. An ultrasonic treatment apparatus according to claim 5, wherein
the cavitation promoting portion is formed to have a shape having a large drag coefficient.

7. An ultrasonic treatment apparatus according to claim 5 used to crush and aspirate the living tissue, further comprising:
an aspiration channel which aspirates the crushed living tissue.

8. An ultrasonic treatment apparatus according to claim 5, wherein the treatment portion is formed to have a shape such that a direction of a velocity of the liquid in the vicinity of the external surface of the cavitation promoting portion corresponds to a direction from the treatment portion toward the tissue in treatment on the tissue in the fluid analysis concerning the ultrasonic oscillations in the liquid.

9. A probe for an ultrasonic treatment apparatus, wherein the probe is connected to a ultrasonic oscillator which generates ultrasonic waves, transmits ultrasonic oscillations generated by the ultrasonic oscillator, and includes a treatment portion which is formed on the probe, treats a living tissue by the transmitted ultrasonic oscillations, and has a cavitation suppressing portion formed to have a such shape that a pressure of a liquid in a vicinity of an external surface of the cavitation suppressing portion is greater than a saturation vapor pressure of the liquid in fluid analysis concerning ultrasonic oscillations in the liquid.

10. A probe for an ultrasonic treatment apparatus, wherein the probe is connected to a ultrasonic oscillator which generates ultrasonic waves, transmits ultrasonic oscillations generated by the ultrasonic oscillator, and includes a treatment portion which is formed on the probe, treats a living tissue by the transmitted ultrasonic oscillations, and has a cavitation promoting portion formed to have such a shape that a pressure of a liquid in a vicinity of an external surface of the cavitation promoting portion is equal to or less than a saturation vapor pressure of the liquid in fluid analysis concerning ultrasonic oscillations in the liquid.

11. A method of manufacturing a probe for ultrasonic treatment apparatus, comprising:
preparing a predetermined shape model for at least part of a treatment portion which treats a living tissue by ultrasonic oscillations;
obtaining, by fluid analysis concerning ultrasonic oscillations in a liquid, a pressure distribution of the liquid with respect to the shape model;
changing a shape of the shape model such that a pressure of at least part of portions where the pressure is equal to or less than a saturation vapor pressure of the liquid in the pressure distribution becomes greater than the saturation vapor pressure of the liquid;
alternately repeating the obtaining the pressure distribution of the liquid and the changing the shape of the shape model; and
forming the treatment portion to have a shape of the shape model.

12. A method according to claim 11, wherein the changing the shape of the shape model includes changing the shape of the shape model such that a drag coefficient is reduced.

13. A method of manufacturing a probe for ultrasonic treatment apparatus, comprising:
preparing a predetermined shape model for at least part of a treatment portion which treats a living tissue by ultrasonic oscillations;
obtaining, by fluid analysis concerning ultrasonic oscillations in a liquid, a pressure distribution of the liquid with respect to the shape model;
changing a shape of the shape model such that a pressure of at least part of portions where the pressure is greater than a saturation vapor pressure of the liquid in the pressure distribution becomes less than the saturation vapor pressure of the liquid;
alternately repeating the obtaining the pressure distribution of the liquid and the changing the shape of the shape model; and
forming the treatment portion to have a shape of the shape model.

14. A method according to claim 13, wherein the changing the shape of the shape model includes changing the shape of the shape model such that a drag coefficient is increased.

15. A method according to claim 13, further comprising:
changing the shape of the shape model such that a direction of a velocity of the liquid in at least part of portions where the pressure is less than the. saturation vapor pressure of the liquid in the pressure distribution corresponds to a direction from the treatment portion toward the tissue in treatment on the tissue.

16. A method of manufacturing an ultrasonic treatment apparatus, comprising a method of manufacturing a probe, including:
preparing a predetermined shape model for at least part of a treatment portion which treats a living tissue by ultrasonic oscillations;
obtaining, by fluid analysis concerning ultrasonic oscillations in a liquid, a pressure distribution of the liquid with respect to the shape model;
changing a shape of the shape model such that a pressure of at least part of portions where the pressure is equal to or less than a saturation vapor pressure of the liquid in the pressure distribution becomes greater than the saturation vapor pressure of the liquid;
alternately repeating the obtaining the pressure distribution of the liquid and the changing the shape of the shape model; and
forming the treatment portion to have a shape of the shape model.

17. A method of manufacturing an ultrasonic treatment apparatus, comprising a method of manufacturing a probe, including:
preparing a predetermined shape model for at least part of a treatment portion which treats a living tissue by ultrasonic oscillations;
obtaining, by fluid analysis concerning ultrasonic oscillations in a liquid, a pressure distribution of the liquid with respect to the shape model;
changing a shape of the shape model such that a pressure of at least part of portions where the pressure is greater than a saturation vapor pressure of the liquid in the pressure distribution becomes less than the saturation vapor pressure of the liquid;
alternately repeating the obtaining the pressure distribution of the liquid and the changing the shape of the shape model; and
forming the treatment portion to have a shape of the shape model.
